(19) United States
(12) Patent Application Publication
(10) Pub. No.: US 2015/0141336 A1
(43) Pub. Date: May 21, 2015

(54) Pancreatic Peptide Compounds and Use

(71) Applicant: Novo Nordisk A/S, Bagsvaerd (DK)

(72) Inventors: Rasmus Joergensen, Maaloev (DK); Soeren Oestergaard, Broenshoej (DK); Lars Ynddal, Jyllinge (DK); Flemming Seier Nielsen, Roskilde (DK)

(21) Appl. No.: 14/402,506

(22) PCT Filed: May 17, 2013
(86) PCT No.: PCT/EP2013/060259

§ 371 Date: Nov. 20, 2014

Related U.S. Application Data
(60) Provisional application No. 61/669,271, filed on Jul. 9, 2012.

(30) Foreign Application Priority Data
May 29, 2012 (EP) 12169817.9

(51) Int. Cl. C07K 14/575

(52) U.S. Cl. CPC C07K 14/575; A61K 38/00
USPC 514/9.7; 530/324

(57) ABSTRACT

The invention relates to novel use of Pancreatic Polypeptides as well as novel Pancreatic Polypeptides and compositions thereof. Such peptides can be used in treating or preventing conditions responsive to Y4 and/or Y5 receptor activation, such as cachexia.

The present invention relates to new PP peptides, compositions thereof and new use of PP peptides for treating and/or preventing conditions responsive to Y4 and/or Y5 receptor activation, such as cachexia.

### BACKGROUND

Pancreatic Polypeptide (PP) is a 36 amino acid peptide hormone released from the pancreas as a response to food-intake. It is a member of the NPY family of peptides and is a high affinity agonist of the Y4 receptor but also have some affinity for the Y5 receptor. PP has been described to inhibit food-intake in rodents and in man but have otherwise only mild gastro-intestinal effects. Due to lack of pronounced physiological effects PP has in several instances been described in the literature as an inert peptide hormone. Patients with PPomas (tumours producing PP) have few clinical signs, and no common clinical sign, despite very high circulating levels of PP. PP has a short half-life of approximately 10 minutes in man. It is known to be DPP-IV substrate and the metabolite PP(3-36) has a half-life of less than 30 minutes in minipigs.

The pharmacological effects of human PP(1-36) or the DPP-IV stabilized peptide PP(2-36) are weak compared to other gastro-intestinal peptide hormones. This may be due to the short half-life of PP, intrinsic properties of the peptide, or a combination of the two.

### SUMMARY

In some embodiments the invention relates to PP peptides for treating and/or preventing conditions responsive to Y4 and/or Y5 receptor activation, wherein said PP peptide comprises an acylation group.

In some embodiments the invention relates to PP peptides comprising an acylation group, wherein
a. said PP peptide is not PP(2-36) substituted with N-epsilon-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl]lysine in position 2, 10, 11, 18, 25, 26, 33, 35 or 36; or wherein
b. said PP peptide is selected from the group consisting of
   i. PP(2-36) comprising said acylation group attached via the N-terminal amino group or any one of positions 3-9, 12-17, 19-24,
   ii. PP(3-36) comprising said acylation group attached via the N-terminal amino group or any one of positions 4-35; and
   iii. APLEPVYPGDNATPEQLARYYKALRHYINLA-Aib-RQRQ.

In some embodiments the invention relates to a composition comprising a PP peptide of the invention and one or more pharmaceutically acceptable excipients.

### BRIEF DESCRIPTION OF DRAWINGS

FIG. 1 shows change of body weight from baseline in C57BI6J DIO male mice (mean±SEM, n=10) after s.c. administration of vehicle, compound AF at 0.03 μmol/kg/day, compound AF at 0.1 μmol/kg every other day, and human PP(2-36), respectively. At day 4, four vehicle mice dosed once with compound AF at 0.03 μmol/kg/day were excluded from the rest of the study.

FIG. 2 shows change of body weight in male C57Bi6J mice on a high fat diet (mean±SEM, n=10-12) after administration of vehicle (s.c. or pump), compound BN (1 μmol/kg/day, s.c.) or reference compound 1 (500 nmol/kg/day, pump).

FIG. 3 shows change of body weight from baseline in male C57B16J mice on a high fat diet (mean±SEM, n=10) after administration of vehicle (s.c. or pump), compound BN (1 μmol/kg/day, s.c.) or reference compound 1 (500 nmol/kg/day, pump).

### DESCRIPTION

Surprisingly, the present inventors have found that PP peptides comprising an acylation group causes increased body weight. Accordingly, in some embodiments the PP peptide of the invention provides increased food intake, increased body weight, and/or increased appetite. Furthermore, in some embodiments PP peptides comprising an acylation group have prolonged in vivo half-life compared to un-acylated PP peptides. In some embodiments the PP peptides of the invention have an improved efficacy, such as increased Y4 and/or Y5 receptor potency, in addition to a prolonged in vivo half-life compared to un-acylated PP peptides, such as PP(2-36) or PP(3-36). PP peptides with a higher efficacy and/or a prolonged in vivo half-life have improved pharmacological properties. Thus, the present inventors have found that acylation of PP peptides not only affect half-life but also the basic pharmacological properties of the PP peptides.

In some embodiments the PP peptides of the invention provides increased selectivity for the Y4 receptor over the Y5 receptor. Increased selectivity for the Y4 receptor over the Y5 receptor would be advantageous for uses of the PP peptide where it is beneficial to avoid the Y5 receptor mediated effects.

In some embodiments a combination of at least two of the features or effects mentioned herein is achieved.

PP peptides

The PP peptide of the invention comprises an acylation group. The acylation group may be covalently attached via the N-terminal amino group, via the amino group of the amidated C-terminal, or via a side chain of an amino acid, such as the epsilon amino group of lysine. In some embodiments the PP peptide comprises an alkyl chain with at least 14 carbon atoms, such as 16, 18 or 20 carbon atoms. In some embodiments the albumin binding side chain is negatively charged at physiological pH. In some embodiments the albumin binding side chain comprises a group which can be negatively charged. In some embodiments the acylation group comprises a distal carboxylic acid group or a distal tetrazole group. In some embodiments the acylation group comprises and a proximal amide group. In some embodiments the acylation group comprises one or more moieties selected from the group consisting of 17-carboxyheptadecanoylamino, 4-carboxybuturylamino and 2-[2-(2-ethoxy)-ethoxy]-acetyl.

In some embodiments the acylation group is 2-(2-{2-[2-(2-{2-[(S)-4-Carboxy-4-({trans-4-[(19-carboxynonadecanoylamino)methyl]cyclohexanecarbonyl}amino)butyrylamino]ethoxy}ethoxy)acetylamino]ethoxy}ethoxy)acetyl. In some embodiments the acylation group is 2-(2-{2-[2-(2-{2-[(S)-4-Carboxy-4-(19-carboxynonadecanoylamino)butyrylamino]ethoxy}ethoxy)acetyl-amino]ethoxy}ethoxy)acetyl. In some embodiments the acylation group is 2-(2-{2-[2-(2-{2-[(S)-4-Carboxy-4-(17-carboxyheptadecanoylamino)butyrylamino]ethoxy}ethoxy)acetyl-amino]ethoxy}ethoxy)acetyl. In some embodiments the acylation group is 2-(2-{2-[2-(2-{2-[(S)-4-Carboxy-4-(15-carboxypentadecanoylamino)butyrylamino]ethoxy}ethoxy)acetyl-amino]ethoxy}ethoxy)acetyl. In some embodiments the acylation group is 2-(2-{2-[2-(2-{2-[(S)-4-Carboxy-4-(hexadecanoylamino)butyrylamino]ethoxy}ethoxy)acetyl-amino]ethoxy}ethoxy)acetyl. In some embodiments the acylation group is [4-(16-(1H-Tetrazol-5-yl)hexadecanoylsulfamoyl)butyryl]ethoxy}ethoxy)acetylamino]ethoxy}ethoxy)acetyl].

In some embodiments the PP peptides of the invention comprise a saturated alkyl chain of at least 16 carbon atoms. In some embodiments the PP peptides of the invention comprise an acylation group with a distal carboxylic acid.

In some embodiments the PP peptide is of human origin. In some embodiments references herein to positions in a PP peptide refers to positions in human PP(1-36). Human PP(1-36) is APLEPVYPGDNATPEQMAQYAADLRRYINMLTRPRQ. Human PP(2-36) and human PP(3-36) is human PP(1-36), wherein the amino acid in position 1 or position 1 and 2, respectively, is deleted. In some embodiments the PP peptide may be derived from vertebrates, such as a mammal, including human, mouse, sheep, goat, cow, or horse.

The term “peptide” as used herein means a compound composed of at least five constituent amino acids connected by peptide bonds. In some embodiments the N-terminus of the peptide is an amino group and/or said C-terminus is a carboxylic acid group. In some embodiments all amino acids in the PP peptide for which the optical isomer is not stated is to be understood to mean the L-isomer. In some embodiments at least one of the amino acids in the PP peptide are D-amino acids. In some embodiments the constituent amino acids of the PP peptide may be selected from at least one of the group of the proteinogenic amino acids encoded by the genetic code and the non-proteinogenic amino acids, such as natural amino acids which are not encoded by the genetic code and synthetic amino acids. As used herein the term “Aib” refers to the amino acid 2-aminoisobutyric acid.

In some embodiments up to 8 amino acids have been substituted, deleted, inserted and/or modified in the PP peptide as compared to PP(1-36). In some embodiments up to 7 amino acids have been substituted, deleted, inserted and/or modified in the PP peptide as compared to PP(1-36). In some embodiments up to 6 amino acids have been substituted, deleted, inserted and/or modified in the PP peptide as compared to PP(1-36). In some embodiments up to 5 amino acids have been substituted, deleted, inserted and/or modified in the PP peptide as compared to PP(1-36). In some embodiments up to 4 amino acids have been substituted, deleted, inserted and/or modified in the PP peptide as compared to PP(1-36). In some embodiments up to 3 amino acids have been substituted, deleted, inserted and/or modified in the PP peptide as compared to PP(1-36). In some embodiments up to 2 amino acids have been substituted, deleted, inserted and/or modified in the PP peptide as compared to PP(1-36). In some embodiments 1 amino acid has been substituted, deleted, inserted and/or modified in the PP peptide as compared to PP(1-36).

In some embodiments the PP peptide exhibits at least 60%, 65%, 70%, 80%, or 90% sequence identity to PP(1-36) over the entire length of PP(1-36). As an example of a method for determination of sequence identity between two peptides, the two peptides [Ala34]PP(1-36) and PP(1-36) are aligned. The sequence identity of [Ala34]PP(1-36) relative to PP(1-36) is given by the number of aligned identical residues minus the number of different residues divided by the total number of residues in PP(1-36). Accordingly, in said example the sequence identity is (36-1)/36.

In some embodiments the PP peptide comprises at least one alteration, such as at least one of substitution, insertion, deletion and/or modification. In some embodiments the PP peptide includes at least one substitution, insertion, deletion and modification of a “non-essential” amino acid residue. A “non-essential” amino acid residue is intended to mean a residue that can be altered, i.e., deleted or substituted, in the sequence of the peptide without abolishing or substantially reducing the activity of said peptide. In some embodiments “activity” of the PP peptide is Y4 receptor potency as determined by a Y4 receptor potency assay, such as Assay (VIII) described herein. The term “substitution” is intended to mean the change of one amino acid in the native sequence with another amino acid. The term “deletion” is intended to mean the removal of one or more amino acids from the reference sequence. The term “insertion” is intended to mean the addition of one or more amino acid into the reference sequence. The term “modification” is intended to mean alterations covalently attached to the side chain of one or more amino acids or the alpha nitrogen atom of one or more amino acid in the reference peptide sequence.

In some embodiments the C-terminal of the PP peptide may be terminated as either an acid or amide. In some embodiments the C-terminal of the PP peptide is an amide.

In some embodiments the PP peptide comprises combinations of two or more changes selected from the group consisting of deletion, insertion, and substitution. In some embodiments the PP peptide comprises one, two or three amino acid substitutions. In some embodiments the PP peptide comprises one, two or three amino acid modifications.

In some embodiments the PP peptide comprises the amino acid sequence of formula (I):

(I)
Xaa₀-Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂-Xaa₁₃-Xaa₁₄-Xaa₁₅-Xaa₁₆-Xaa₁₇-Xaa₁₈-
Xaa₁₉-Xaa₂₀-Xaa₂₁-Xaa₂₂-Xaa₂₃-Xaa₂₄-Xaa₂₅-Xaa₂₆-Xaa₂₇-
Xaa₂₈-Xaa₂₉-Xaa₃₀-Xaa₃₁-Xaa₃₂-Xaa₃₃-Xaa₃₄-Xaa₃₅-Xaa₃₆

wherein

Xaa₀ is Lys or absent,
Xaa₁ is Ala, Gly, Ser, Thr, Lys, or absent,
Xaa₂ is Pro, Lys, or absent,
Xaa₃ is Leu, Pro, Ile, Ser, Lys, or absent,
Xaa₄ is Glu, or Lys,
Xaa₅ is Pro, Ala, or Lys,
Xaa₆ is Val, or Lys,
Xaa₇ is Tyr, or Lys,
Xaa₈ is Pro, Ala, or Lys,
Xaa₉ is Gly, Ala, or Lys,
Xaa₁₀ is Asp, Asn, Glu, Gln, or Lys,
Xaa₁₁ is Asn, Asp, or Lys,
Xaa₁₂ is Ala, or Lys,
Xaa₁₃ is Thr, or Lys,
Xaa₁₄ is Pro, or Lys,
Xaa₁₅ is Glu, or Lys,
Xaa₁₆ is Gln, or Lys,
Xaa₁₇ is Leu, Met, Val, Ile, or Lys,
Xaa₁₈ is Ala, or Lys,
Xaa₁₉ is Gin, or Lys,
Xaa₂₀ is Tyr, Phe, or Lys,
Xaa₂₁ is Ala, or Lys,
Xaa₂₂ is Ala, or Lys,
Xaa₂₃ is Asp, or Lys,
Xaa₂₄ is Leu, Val, Ile, or Lys,
Xaa₂₅ is Arg, or Lys,
Xaa₂₆ is Arg, His, or Lys,
Xaa₂₇ is Tyr, Phe, or Lys,
Xaa₂₈ is Ile, Val, Leu, or Lys,
Xaa₂₉ is Asn, Gin, or Lys, or Lys,
Xaa₃₀ is Met, Leu, Val, Ile, or Lys,
Xaa₃₁ is Leu, Val, Ile, or Lys,
Xaa₃₂ is Ser, Thr, or Lys,
Xaa₃₃ is Arg, Lys, or Lys,
Xaa₃₄ is Pro, Gin, Asn, His, or Lys,
Xaa₃₅ is Arg or Lys, or Lys,
Xaa₃₆ is Tyr, or Lys.

In some embodiments Xaa₀ is absent. In some embodiments Xaa₁ is not Ala. In some embodiments Xaa₂ is not Pro. In some embodiments Xaa₃ is not Leu. In some embodiments Xaa₄ is not Glu. In some embodiments Xaa₅ is not Pro. In some embodiments Xaa₆ is not Val. In some embodiments Xaa₇ is not Tyr. In some embodiments Xaa₈ is not Pro. In some embodiments Xaa₉ is not Gly. In some embodiments Xaa₁₀ is not Asp. In some embodiments Xaa₁₁ is not Asn. In some embodiments Xaa₁₂ is not Ala. In some embodiments Xaa₁₃ is not Thr. In some embodiments Xaa₁₄ is not Pro. In some embodiments Xaa₁₅ is not Glu. In some embodiments Xaa₁₆ is not Gln. In some embodiments Xaa₁₇ is not Met. In some embodiments Xaa₁₈ is not Ala. In some embodiments Xaa₁₉ is not Gln. In some embodiments Xaa₂₀ is not Tyr. In some embodiments Xaa₂₁ is not Ala. In some embodiments Xaa₂₂ is not Ala. In some embodiments Xaa₂₃ is not Asp. In some embodiments Xaa₂₄ is not Leu. In some embodiments Xaa₂₅ is not Arg. In some embodiments Xaa₂₆ is not Arg. In some embodiments Xaa₂₇ is not Tyr. In some embodiments Xaa₂₈ is not Ile. In some embodiments Xaa₂₉ is not Asn. In some embodiments Xaa₃₀ is not Met. In some embodiments Xaa₃₁ is not Leu. In some embodiments Xaa₃₂ is not Thr. In some embodiments Xaa₃₃ is not Arg. In some embodiments Xaa₃₄ is not Pro. In some embodiments Xaa₃₅ is not Arg. In some embodiments Xaa₃₆ is not Tyr.

In some embodiments Xaa₁ is absent. In some embodiments Xaa₁ and Xaa₂ are absent. In some embodiments Xaa₀ is Lys. In some embodiments Xaa₃ is Pro or hydroxyproline. In some embodiments Xaa₁ is Ala. In some embodiments Xaa₂ is Pro. In some embodiments Xaa₃ is Leu. In some embodiments Xaa₄ is Glu. In some embodiments Xaa₅ is Pro. In some embodiments Xaa₆ is Val. In some embodiments Xaa₇ is Tyr. In some embodiments Xaa₈ is Pro. In some embodiments Xaa₉ is Gly. In some embodiments Xaa₁₀ is Asp. In some embodiments Xaa₁₁ is Asn. In some embodiments Xaa₁₂ is Ala. In some embodiments Xaa₁₃ is Thr. In some embodiments Xaa₁₄ is Pro. In some embodiments Xaa₁₅ is Glu. In some embodiments Xaa₁₆ is Gln. In some embodiments Xaa₁₇ is Met. In some embodiments Xaa₁₈ is Ala. In some embodiments Xaa₁₉ is Gln. In some embodiments Xaa₂₀ is Tyr. In some embodiments Xaa₂₁ is Ala. In some embodiments Xaa₂₂ is Ala. In some embodiments Xaa₂₃ is Asp. In some embodiments Xaa₂₄ is Leu. In some embodiments Xaa₂₅ is Arg. In some embodiments Xaa₂₆ is Arg. In some embodiments Xaa₂₇ is Tyr. In some embodiments Xaa₂₈ is Ile. In some embodiments Xaa₂₉ is Asn. In some embodiments Xaa₃₀ is Met. In some embodiments Xaa₃₁ is Leu. In some embodiments Xaa₃₂ is Thr. In some embodiments Xaa₃₃ is Arg. In some embodiments Xaa₃₄ is Pro. In some embodiments Xaa₃₅ is Arg. In some embodiments Xaa₃₆ is Tyr. In some embodiments Xaa₂₅ is Ala.

The PP peptides of the invention includes compounds A to BM, which as defined in Table 1 consist of the PP(2-36) or PP(3-36) peptide, wherein the amino acid in the position defined in Table 1 is substituted with the following modified lysine:

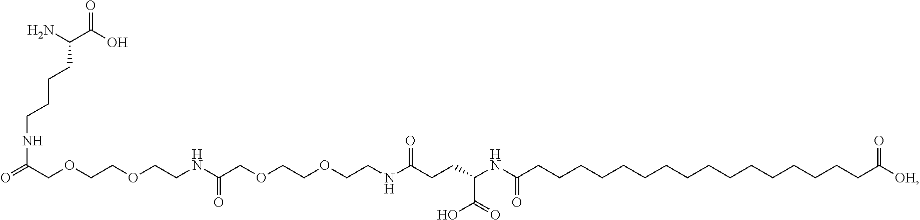 [SMILES: N[C@@H](CCCCNC(=O)COCCOCCNC(=O)COCCOCCNC(=O)CC[C@H](NC(=O)CCCCCCCCCCCCCCCCC(=O)O)C(=O)O)C(=O)O]

except for the compounds AF and BM in which the group [2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl] is covalently attached to the N-terminal amino group (referred to as “Nα” in Table 1).

TABLE 1
PP peptides of the invention

| Compound | Based on PP peptide | Acylation position |
|---|---|---|
| A | PP(2-36) | 34 |
| B | PP(2-36) | 32 |
| C | PP(2-36) | 31 |
| D | PP(2-36) | 30 |
| E | PP(2-36) | 29 |
| F | PP(2-36) | 28 |
| G | PP(2-36) | 27 |
| H | PP(2-36) | 26 |
| I | PP(2-36) | 25 |
| J | PP(2-36) | 24 |
| K | PP(2-36) | 23 |
| L | PP(2-36) | 22 |
| M | PP(2-36) | 21 |
| N | PP(2-36) | 20 |
| O | PP(2-36) | 19 |
| P | PP(2-36) | 18 |
| Q | PP(2-36) | 17 |
| R | PP(2-36) | 16 |
| S | PP(2-36) | 15 |
| T | PP(2-36) | 14 |
| U | PP(2-36) | 13 |
| V | PP(2-36) | 12 |
| W | PP(2-36) | 11 |
| X | PP(2-36) | 10 |
| Y | PP(2-36) | 9 |
| Z | PP(2-36) | 8 |
| AA | PP(2-36) | 7 |
| AB | PP(2-36) | 6 |
| AC | PP(2-36) | 5 |
| AD | PP(2-36) | 4 |
| AE | PP(2-36) | 3 |
| AF | PP(2-36) | Nα |
| AG | PP(3-36) | 35 |
| AH | PP(3-36) | 34 |
| AI | PP(3-36) | 33 |
| AJ | PP(3-36) | 32 |
| AK | PP(3-36) | 31 |
| AL | PP(3-36) | 30 |
| AM | PP(3-36) | 29 |
| AN | PP(3-36) | 28 |
| AO | PP(3-36) | 27 |
| AP | PP(3-36) | 26 |
| AQ | PP(3-36) | 25 |
| AR | PP(3-36) | 24 |
| AS | PP(3-36) | 23 |
| AT | PP(3-36) | 22 |
| AU | PP(3-36) | 21 |
| AV | PP(3-36) | 20 |
| AW | PP(3-36) | 19 |
| AX | PP(3-36) | 18 |
| AY | PP(3-36) | 17 |
| AZ | PP(3-36) | 16 |
| BA | PP(3-36) | 15 |
| BB | PP(3-36) | 14 |
| BC | PP(3-36) | 13 |
| BD | PP(3-36) | 12 |
| BE | PP(3-36) | 11 |
| BF | PP(3-36) | 10 |
| BG | PP(3-36) | 9 |
| BH | PP(3-36) | 8 |
| BI | PP(3-36) | 7 |
| BJ | PP(3-36) | 6 |
| BK | PP(3-36) | 5 |
| BL | PP(3-36) | 4 |
| BM | PP(3-36) | Nα |

In some embodiments the PP peptide is compound A. In some embodiments the PP peptide is compound B. In some embodiments the PP peptide is compound C. In some embodiments the PP peptide is compound D. In some embodiments the PP peptide is compound E. In some embodiments the PP peptide is compound F. In some embodiments the PP peptide is compound G. In some embodiments the PP peptide is compound H. In some embodiments the PP peptide is compound i. In some embodiments the PP peptide is compound J. In some embodiments the PP peptide is compound K. In some embodiments the PP peptide is compound L. In some embodiments the PP peptide is compound M. In some embodiments the PP peptide is compound N. In some embodiments the PP peptide is compound O. In some embodiments the PP peptide is compound P. In some embodiments the PP peptide is compound Q. In some embodiments the PP peptide is compound R. In some embodiments the PP peptide is compound S. In some embodiments the PP peptide is compound S. In some embodiments the PP peptide is compound T. In some embodiments the PP peptide is compound U. In some embodiments the PP peptide is compound V. In some embodiments the PP peptide is compound W. In some embodiments the PP peptide is compound X. In some embodiments the PP peptide is compound Y. In some embodiments the PP peptide is compound Z. In some embodiments the PP peptide is compound AA. In some embodiments the PP peptide is compound AB. In some embodiments the PP peptide is compound AC. In some embodiments the PP peptide is compound AD. In some embodiments the PP peptide is compound AE. In some embodiments the PP peptide is compound AF. In some embodiments the PP peptide is compound AG. In some embodiments the PP peptide is compound AH. In some embodiments the PP peptide is compound Al. In some embodiments the PP peptide is compound AJ. In some embodiments the PP peptide is compound AK. In some embodiments the PP peptide is compound AL. In some embodiments the PP peptide is compound AM. In some embodiments the PP peptide is compound AN. In some embodiments the PP peptide is compound AO. In some embodiments the PP peptide is compound AP. In some embodiments the PP peptide is compound AQ. In some embodiments the PP peptide is compound AR. In some embodiments the PP peptide is compound AS. In some embodiments the PP peptide is compound AT. In some embodiments the PP peptide is compound AU. In some embodiments the PP peptide is compound AV. In some embodiments the PP peptide is compound AW. In some embodiments the PP peptide is compound AX. In some embodiments the PP peptide is compound AY. In some embodiments the PP peptide is compound AZ. In some embodiments the PP peptide is compound BA. In some embodiments the PP peptide is compound BB. In some embodiments the PP peptide is compound BC. In some embodiments the PP peptide is compound BD. In some embodiments the PP peptide is compound BE. In some embodiments the PP peptide is compound BF. In some embodiments the PP peptide is compound BG. In some embodiments the PP peptide is compound BH. In some embodiments the PP peptide is compound BI. In some embodiments the PP peptide is compound BJ. In some embodiments the PP peptide is compound BK. In some embodiments the PP peptide is compound BL. In some embodiments the PP peptide is compound BM. In some embodiments the PP peptide is compound BN. The structure of compound BN is:

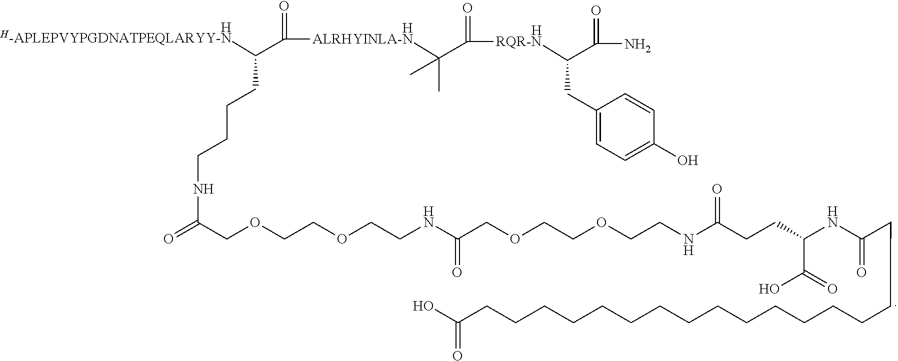

Reference compound 1 is the non-acylated version of compound BN, i.e. wherein the modified lysine in position 22 of compound BN is a lysine residue.

In some embodiments the acylation group comprises a saturated alkyl chain with at least 14 carbon atoms, such as 16-20 carbon atoms, wherein said alkyl chain optionally comprises a distal carboxylic acid or a distal tetrazole group.

In some embodiments the acylation group comprises an 8-amino-3,6-dioxaoctanoic acid (Oeg) molecule.

In some embodiments the acylation group is covalently attached to the N-terminal amino group or the epsilon amino group of a lysine.

In some embodiments the PP peptide comprises PP(3-36), PP(2-36), or PP(1-36). In some embodiments the PP peptide comprises PP(3-36), PP(2-36), or PP(1-36) with no more than 5 or 4, such as no more than 3, 2 or 1, amino acids substitutions, deletions and/or additions.

In some embodiments the acylation group comprises the moiety [2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl].

In some embodiments the PP peptide is selected from the group consisting of
a. PP(2-36) comprising said acylation group attached via the N-terminal amino group or any one of positions 3-9, 12-17, 19-24, 27-32 or 34;
b. PP(3-36) comprising said acylation group attached via the N-terminal amino group or any one of positions 4-35; and
c. APLEPVYPGDNATPEQLARYYKALRHYINLA-Aib-RQRQ.

In some embodiments the PP peptide is selected from the group consisting of compound A to compound BM and

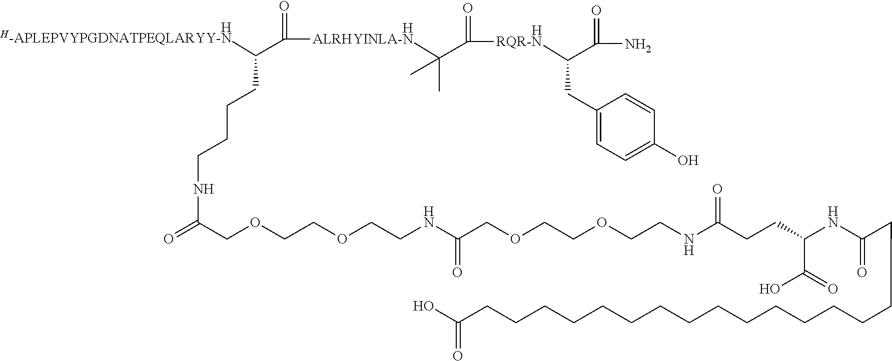

(compound BN).

In some embodiments the PP peptide comprises an acylation group, wherein
a. said PP peptide is not PP(2-36) substituted with N-epsilon-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl]lysine in position 2, 10, 11, 18, 25, 26, 33, 35 or 36; or wherein
b. said PP peptide is selected from the group consisting of
   i. PP(2-36) comprising said acylation group attached via the N-terminal amino group or any one of positions 3-9, 12-17, 19-24,
   ii. PP(3-36) comprising said acylation group attached via the N-terminal amino group or any one of positions 4-35; and
   iii. APLEPVYPGDNATPEQLARYYKALRHYINLA-Aib-RQRQ.

In some embodiments the PP peptide has a half-life of at least 2 times, such as at least 3, 4, 5 or 8 times, the half-life of PP(1-36). In some embodiments the PP peptide has a half-life of at least 7 h, such as at least 10, 20, 40 or 40 h, wherein the half-life is determined by Assay (II) described herein.

In some embodiments the PP peptide has a Y4 and/or Y5 receptor potency of <100 nM, such as <50 nM, <20 nM, or <10 nM, as determined by Assay (VIII) and/or (IX), respectively.

In some embodiments a therapeutically effective dosage of said PP peptide is administered once daily or less often, such as once weekly or less often.

In some embodiments the a therapeutically effective dosage of said PP peptide is administered for a period of at least 2 days, such as at least 3 days or at least 4 days.

### Compositions

In some embodiments the present invention provides a pharmaceutical composition comprising the PP peptide and one or more excipients. In some embodiments the pharmaceutical composition comprises the PP peptide in a concentration from 0.1 mg/ml to 25 mg/ml. In some embodiments the pharmaceutical composition has a pH from 3.0 to 9.0. The formulation may further comprise at least one component selected from the group consisting of a buffer system, preservative(s), tonicity agent(s), chelating agent(s), stabilizer(s) and surfactant(s). In some embodiments the composition comprising excipients selected from the group consisting of a buffer, a preservative, and optionally a tonicity modifier and/or a stabilizer.

### Indications

The PP peptides and compositions containing them are also useful in the manufacture of a medicament for therapeutic applications mentioned herein. In some embodiments the invention relates to the use of at least one PP peptide for the preparation of a medicament. In some embodiments a method of treating a disease, condition or disorder modulated by a Y4 receptor agonist using the PP peptide thereof is provided. In some embodiments a method of treating a disease, condition or disorder modulated by a Y5 receptor agonist using the PP peptide is provided. In some embodiments the invention relates to a method of treating and/or preventing conditions responsive to Y4 and/or Y5 receptor activation. In some embodiments the invention relates to a method of increasing food intake, increasing body weight and/or increasing appetite.

In some embodiments the PP peptide of the invention is for use in a condition selected from the group consisting of cachexia or any form or anorexia.

In some embodiments the PP peptide of the invention is for the use in a condition characterized by damage to the intestine, such as chemotherapy-induced diarrhoea, ulcerative colitis, inflammatory bowel disease, bowel atrophy, loss bowel mucosa, and/or loss of bowel mucosal function.

In some embodiments the PP peptide of the invention is for the use in treatment of any form of diabetes mellitus, insulin resistance or any condition characterized by insulin resistance or glucose intolerance. In some embodiments the PP peptide of the invention is an insulin sensitizer.

As used herein, the term “therapeutically effective amount” of a compound refers to an amount sufficient to cure, alleviate, or partially arrest the clinical manifestations of a given disease and/or its complications with respect to appropriate control values determined prior to treatment or in a vehicle-treated group. An amount adequate to accomplish this is defined as a “therapeutically effective amount”. Effective amounts for each purpose will depend on the severity of the disease or injury, as well as on the weight and general state of the subject. It will be understood that determination of an appropriate dosage may be achieved using routine experimentation, by constructing a matrix of values and testing different points in the matrix, all of which is within the level of ordinary skill of a trained physician or veterinarian.

### Syntheses

PP peptides of the invention may be synthesized by standard solid phase peptide synthesis (SPPS), using either an automated peptide synthesizer, or traditional bench synthesis. The solid support can be, e.g., Tentagel S RAM, chlorotrityl (CI) or Wang (OH) resin, all of which are readily available commercially. The active amino or hydroxyl groups of those resins react readily with the carboxyl group of an N-Fmoc amino acid, thereby covalently binding it to the polymer via a linkage to a linker attached to the resin. The resin-bound Fmoc-amino acid may be deprotected by exposure to a mixture of 20% piperidine in N-methylpyrrolidinone (NMP) which readily cleaves the Fmoc-group. The subsequent amino acid is coupled using a coupling reagent and followed by another deprotection of the Fmoc-group. Examples of reagents facilitating the coupling of incoming amino acids to the resin-bound amino acid chain are: diisopropylcarbodiimide (DIC), tetramethyluroniumhexafluorophosphate (HATU), O-(1H-benzotriazole-1-yl)-N,N,N\N′-tetramethyluroniumhexafluorophosphate (HBTU), O-(1H-benzotriazole-1-yl)-N,N,N′,N′-tetramethyluroniumtetrafluoroborate (TBTU), 1H-hydroxybenzotriazole (HOBt). The SPPS is continued a stepwise manner until the desired sequence is obtained. At the end of the synthesis, the resin-bound protected peptide is deprotected cleaving the protection groups on the side chains and also cleaving the peptide from the resin. This is done with trifluoroacetic acid (TFA) containing scavengers, such as triisopropylsilane (TIPS). The peptide is then precipitated in diethylether and isolated. Peptide synthesis by solution chemistry rather than solid phase chemistry is also feasible.

It may be desirable to purify the PP peptides generated by the present invention. Peptide purification techniques are well known to those of skill in the art. These techniques involve, at one level, the crude fractionation of the cellular milieu to peptide and non-peptide fractions. Having separated the peptide from other proteins, the peptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography, polyacrylamide gel electrophoresis, and isoelectric focusing. A particularly efficient method of purifying peptides is reverse phase HPLC, followed by characterization of purified product by liquid chromatography/mass spectrometry (LC/MS) and Matrix-Assisted Laser Desorption Ionization (MALDI) mass spectrometry. Additional confirmation of purity is obtained by determining amino acid analysis.

Certain embodiments of the present invention concern the purification, and in particular embodiments, the substantial purification, of a peptide, including the PP peptide according to the invention. The term “purified peptide” as used herein, is intended to refer to a composition, isolatable from other components, wherein the peptide is purified to any degree relative to its naturally obtainable state. A purified peptide therefore also refers to a peptide, free from the environment in which it may naturally occur. Generally, “purified” will refer to a peptide composition that has been subjected to fractionation to remove various other components, and which composition substantially retains its expressed biological activity. Where the term “substantially purified” is used, this designation will refer to a composition in which the peptide forms the major component of the composition, such as constituting about 50%, about 60%, about 70%, about 80%, about 90%, about 95% or more of the peptides in the composition.

Various techniques suitable for use in peptide purification will be well known to those of skill in the art. These include, e.g., precipitation with ammonium sulphate, PEG, antibodies, and the like; heat denaturation, followed by centrifugation; chromatography steps, such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the peptides always be provided in their most purified state. Indeed, it is contemplated that less substantially purified products will have utility in certain embodiments. Partial purification may be accomplished by using fewer purification steps in combination, or by utilizing different fopins of the same general purification scheme. For example, it is appreciated that a cation-exchange column chromatography performed, utilizing an HPLC apparatus, will generally result in a greater “-fold” purification than the same technique utilizing a low pressure chromatography system. Methods exhibiting a lower degree of relative purification may have advantages in total recovery of protein product, or in maintaining the activity of an expressed protein.

One may optionally purify and isolate PP peptides of the invention from other components obtained in the process. Methods for purifying a peptide can be found in U.S. Pat. No. 5,849,883. These documents describe specific exemplary methods for the isolation and purification of G-CSF compositions that may be useful in isolating and purifying PP peptides of the invention. A person skilled in the art would be well aware of numerous purification techniques that may be used to purify PP peptides of the invention from a given source.

Also it is contemplated that a combination of anion exchange and immunoaffinity chromatography may be employed to produce purified compositions of PP peptides.

### Embodiments of the Invention

Non-limiting embodiments of the invention are:

1. A PP peptide for treating and/or preventing conditions responsive to Y4 and/or Y5 receptor activation, wherein said PP peptide comprises an acylation group.
2. A PP peptide according to, embodiments, wherein said treating and/or preventing provides increased food intake, increased body weight and/or increased appetite.
3. A PP peptide according to, embodiments, wherein said condition is cachexia.
4. A PP peptide according to, embodiments, wherein said condition is a condition characterized by damage to the intestine, such as chemotherapy-induced diarrhoea, ulcerative colitis, inflammatory bowel disease, bowel atrophy, loss bowel mucosa, and/or loss of bowel mucosal function.
5. A PP peptide according to, embodiments, wherein said acylation group comprises a saturated alkyl chain with at least 14 carbon atoms, such as 16-20 carbon atoms, wherein said alkyl chain optionally comprises a distal carboxylic acid or a distal tetrazole group.
6. A PP peptide according to, embodiments, wherein said acylation group comprises an 8-amino-3,6-dioxaoctanoic acid (Oeg) molecule.
7. A PP peptide according to, embodiments, wherein said acylation group is covalently attached to the N-terminal amino group or the epsilon amino group of a lysine.
8. A PP peptide according to, embodiments, wherein said PP peptide comprises PP(3-36), PP(2-36), or PP(1-36), and wherein said PP(3-36), PP(2-36), or PP(1-36) comprises no more than 5 or 4, such as no more than 3, 2 or 1, amino acids substitutions, deletions and/or additions.
9. A PP peptide according to, embodiments, wherein said acylation group comprises the moiety [2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl].
10. A PP peptide according to, embodiments, wherein said PP peptide is selected from the group consisting of
a. PP(2-36) comprising said acylation group attached via the N-terminal amino group or any one of positions 3-9, 12-17, 19-24, 27-32 or 34;
b. PP(3-36) comprising said acylation group attached via the N-terminal amino group or any one of positions 4-35; and
c. APLEPVYPGDNATPEQLARYYKALRHYINLA-Aib-RQRQ.
   11. A PP peptide according to, embodiments, wherein PP peptide is selected from the group consisting of compound A to compound BM and

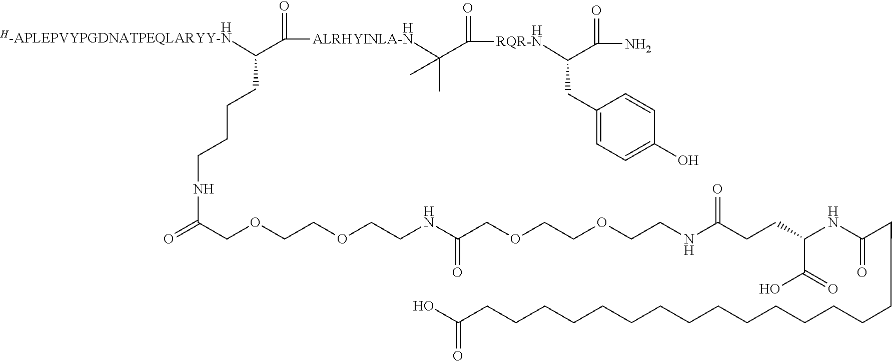

(compound BN).
12. A PP peptide according to, embodiments, wherein said PP peptide has a half-life of at least 2 times, such as at least 3, 4, 5 or 8 times, the half-life of PP(1-36) or wherein said PP peptide has a half-life of at least 7 h, such as at least 10, 20, 40 or 40 h, wherein the half-life is determined by Assay (II) described herein.
13. A PP peptide according to, embodiments, wherein said PP peptide has a Y4 and/or Y5 receptor potency of <100 nM, such as <50 nM, <20 nM, or <10 nM, as determined by Assay (VIII) and/or (IX), respectively.
14. A PP peptide according to, embodiments, wherein a therapeutically effective dosage of said PP peptide is administered once daily or less often, such as once weekly or less often.
15. A PP peptide according to, embodiments, wherein a therapeutically effective dosage of said PP peptide is administered for a period of at least 2 days, such as at least 3 days or at least 4 days.
16. A PP peptide comprising an acylation group, wherein
a. said PP peptide is not PP(2-36) substituted with N-epsilon-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl]lysine in position 2, 10, 11, 18, 25, 26, 33, 35 or 36; or wherein
b. said PP peptide is selected from the group consisting of
   i. PP(2-36) comprising said acylation group attached via the N-terminal amino group or any one of positions 3-9, 12-17, 19-24, 27-32 or 34;
   ii. PP(3-36) comprising said acylation group attached via the N-terminal amino group or any one of positions 4-35; and
   iii. APLEPVYPGDNATPEQLARYYKALRHYINLA-Aib-RQRQ.
      17. A PP peptide according to embodiment 16, wherein said acylation group comprises a saturated alkyl chain with at least 14 carbon atoms, such as 16-20 carbon atoms, wherein said alkyl chain optionally comprises a distal carboxylic acid or a distal tetrazole group.
      18. A PP peptide according to any one of embodiments 16-17, wherein said acylation group comprises an 8-amino-3,6-dioxaoctanoic acid (Oeg) molecule.
      19. A PP peptide according to any one of embodiments 16-18, wherein said acylation group is covalently attached to the N-terminal amino group or the epsilon amino group of a lysine.
      20. A PP peptide according to any one of embodiments 16-19, wherein PP peptide is selected from the group consisting of compound A to compound BM and

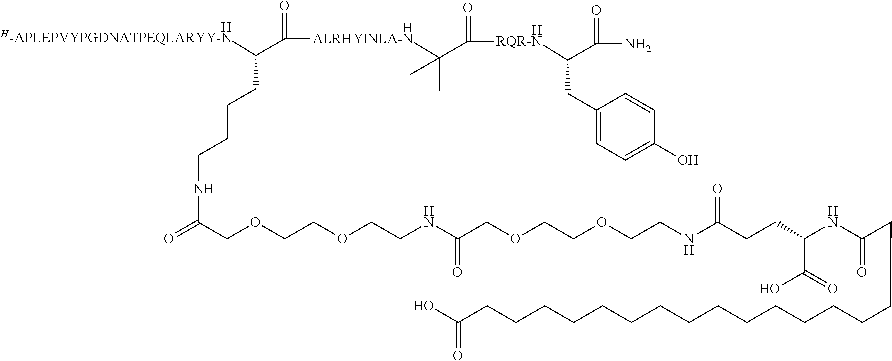

(compound BN).
21. A PP peptide according to any one of embodiments 16-20, wherein said PP peptide is as defined in any one of embodiments 1-15.
22. A composition comprising a PP peptide as defined in any one of embodiment 16-21 and one or more pharmaceutically acceptable excipients.

### EXAMPLES

### Materials and Methods

### List of Abbreviations

Abbreviations used herein:

Abbreviation Meaning
r.t: Room temperature
DIPEA: Diisopropylethylamine
H₂O: Water
CH₃CN: Acetonitrile
DMF: N,N-dimethylformamide
HBTU: 2-(1H-Benzotriazol-1-yl-)-1,1,3,3 tetramethyluroniumhexafluorophosphate
Fmoc: 9H-fluoren-9-ylmethoxycarbonyl
Boc: tertbutyloxycarbonyl
OtBu: tert butyl ester
tBu: tert butyl
Trt: Triphenylmethyl
Pmc: 2,2,5,7,8-Pentamethyl-chroman-6-sulfonyl
Dde: 1-(4,4-Dimethyl-2,6-dioxocyclohexylidene)ethyl
ivDde: 1-(4,4-Dimethyl-2,6-dioxocyclohexylidene)-3-methylbutyl
Mtt: 4-methyltrityl
Mmt: 4-methoxytrityl
DCM: Dichloromethane
TIPS: triisopropylsilane
TFA: trifluoroacetic acid
Et₂O: Diethylether
NMP: 1-Methyl-pyrrolidin-2-one
DIPEA: Diisopropylethylamine
HOAt: 1-Hydroxy-7-azabenzotriazole
HOBt: 1-Hydroxybenzotriazole
DIC: Diisopropylcarbodiimide
MW: Molecular weight

### General Methods of Preparation

Synthesis of Resin Bound Peptide, SPPS Method:

The protected peptidyl resin was synthesized according to the Fmoc strategy on a Prelude Solid Phase Peptide Synthesizer from Protein Technologies in 0.25 mmol scale using DIC and HOAt mediated couplings in NMP. The starting resin used for the synthesis of the peptide amides was Rink-Amide resin. The protected amino acid derivatives used were standard Fmoc-amino acids (supplied from e.g. Anaspec, Bachem, Iris Biotech, or Novabiochem). The epsilon amino group of lysines to be acylated were protected with Mtt. The synthesis of the peptides may in some cases be improved by the use of dipeptides, e.g., pseudoprolines from Novabiochem, Fmoc-Ser(tbu)-ψSer(Me,Me)-OH, see e.g. catalogue from Novobiochem 2002/2003 or newer version, or W. R. Sampson (1999), J. Pep. Sci. 5, 403.

Procedure for Cleaving the Peptide of the Resin:

After synthesis the resin was washed with DCM and dried, and the peptide was cleaved from the resin by a 2 hour treatment with TFA/TIPS/water (92.5/5/2.5), TFA/water/TIPS/thioanisol (90/3/5/2) or TFA/TIPS (95/5) followed by precipitation with diethylether. The peptide was redissolved in 30% acetic acid or similar solvent and purified by standard RP-HPLC on a C18 column using acetonitrile/TFA. The identity of the peptide was confirmed by MALDI-MS.

Procedure for Removal of Mtt-Protection:

The resin was placed in a syringe and treated with hexafluroisopropanol for 2×10 min to remove the Mtt group. The resin was then washed with DCM and NMP as described above.

Procedure for Attachment of Side Chains to Lysine Residue:

The albumin binding residue A-B-C-D, A-C-D, A-B-C or A-B can be attached to the peptide either by acylation to resin bound peptide or acylation in solution to the unprotected peptide using standard acylating reagent, such as but not limited to DIC, HOBt/DIC, HOAt/DIC, or HBTU.

Procedure for Removal of Fmoc-Protection:

The resin (0.25 mmol) was placed in a filter flask in a manual shaking apparatus and treated with N-methyl pyrrolidone/methylene chloride (1:1) (2×20 ml) and with N-methyl pyrrolidone (1×20 ml), a solution of 20% piperidine in N-methyl pyrrolidone (3×20 ml, 10 min each). The resin was washed with N-methyl pyrrolidone (2×20 ml), N-methyl pyrrolidone/Methylene chloride (1:1) (2×20 ml) and methylene chloride (2×20 ml).

General Procedure for N-Terminal Capping:

The resin (0.25 mmol) was placed in a filter flask in a manual shaking apparatus and treated with N-methyl pyrrolidone/methylene chloride (1:1) (2×20 ml) and a solution of e.g. propionic acid anhydride/dichloromethan (1:1) (2×20 ml) was added. The resin was washed with N-methyl pyrrolidone (2×20 ml), N-methyl pyrrolidone/Methylene chloride (1:1) (2×20 ml) and methylene chloride (2×20 ml).

### General Methods of Detection and Characterisation

The PP peptide was optionally purified by UPLC.

### Example 1

### Preparation of PP Peptides

The following PP peptides were prepared using the methods described in the section General Methods of Preparation:

Compound A, crude sample

Compound B, crude sample

Compound C, crude sample

Compound D, crude sample

Compound E, crude sample

Compound F, crude sample

Compound G, crude sample

Compound H, purified sample

Compound I, purified sample

Compound J, crude sample

Compound K, crude sample

Compound L, crude sample

Compound M, crude sample

Compound N, crude sample

Compound O, crude sample

Compound P, purified sample

Compound Q, crude and purified sample

Compound R, crude and purified sample

Compound S, crude sample

Compound T, crude sample

Compound U, crude sample

Compound V, crude sample

Compound W, crude sample

Compound X, crude sample

Compound Y, crude sample

Compound Z, crude sample

Compound AA, crude sample

Compound AB, crude sample

Compound AC, crude sample

Compound AD, crude sample

Compound AE, crude sample

Compound AF, crude and purified sample

Compound AG, crude sample

Compound AH, crude sample

Compound Al, crude sample

Compound AJ, crude sample

Compound AK, crude sample

Compound AL, crude sample

Compound AM, crude sample

Compound AN, crude sample

Compound AO, crude sample

Compound AP, crude sample

Compound AQ, crude sample

Compound AR, crude sample

Compound AS, crude sample

Compound AT, crude sample

Compound AU, crude sample

Compound AV, crude sample

Compound AW, crude sample

Compound AX, purified sample

Compound AY, crude sample

Compound AZ, crude sample

Compound BA, crude sample

Compound BB, crude sample

Compound BC, crude sample

Compound BD, crude sample

Compound BE, crude sample

Compound BF, crude sample

Compound BG, crude sample

Compound BH, crude sample

Compound BI, crude sample

Compound BJ, crude sample

Compound BK, crude sample

Compound BM, crude and purified sample

Compound BN, purified sample

Reference compound 1 (non-acylated variant of compound BN), purified sample

### Biological Assays

The utility of the PP peptides of the present invention as pharmaceutically active agents in the reduction of weight gain and treatment of obesity in mammals (such as humans), may be demonstrated by the activity of the agonists in conventional assays and in the in vitro and in vivo assays described below. Such assays also provide a means whereby the activities of the PP peptides of this invention can be compared with the activities of known compounds, such as human PP(1-36).

### Assay (I): In Vitro DPP-IV Stability

10 μM of peptide was incubated with DPP-IV (2 μg/ml) at 37° C. in a HEPES buffer to which 0.005% Tween20 and 0.001% BSA were added. Aliqouts of sample was taken at 3, 8, 15, 30, 60, 120 and 180 min and three volumes of ethanol were added to stop the reaction. The samples were analysed by LC-MS for parent peptide and for metabolite formation.

Assay (II): PK i.v.minipig

An assay useful for measuring the pharmacokinetic (PK) profile of the PP peptide is the following mini-pig PK assay.

Five male Göttingen mini-pigs weighing approximately 18 to 22 kg from Ellegaard Göttingen Minipigs NS, Denmark are included in the study. The mini-pigs have two central venous catheters inserted which are used for intra venous (i.v.) dosing and blood sampling. The test compound is dissolved in 50 mM K2HPO4, 0.05% tween 80, pH=8.0 to a concentration of 180 nmol/ml. For comparison a control compound, such as human PP(1-36), may be administered. The pigs are dosed with 6 nmol test compound/kg body weight. Blood samples are taken at the following time points: pre-dose, 30 minutes, 1, 2, 4, 8, 24, 48, 72, 96, 120, 168 and 240 hours post dosing. The blood samples were collected into test tubes containing EDTA buffer for stabilization and kept on ice for max. 20 minutes before centrifugation. The centrifugation procedure to separate plasma may be: 4° C., approx. 2500 g for 10 minutes. Plasma is collected and immediately transferred to Micronic tubes stored at −20° C. until assayed.

### Quantitative Assay for Plasma Samples—In Vivo Half Life

The test compounds were assayed in plasma by Turbulent Flow Chromatography coupled to Liquid Chromatography with subsequent Tandem Mass Spectrometric Detection (TFC/LC/MS/MS). Positive mode ionization and Multiple Reaction Monitoring (MRM) of a multiple protonated species fragmented to a singly charged ion was employed for selectivity. The selectivity of the method allows multiple compounds to be quantified in one sample.

The concentrations of the test compound in samples of unknown concentration were calculated using the peak area as a function of amount. Calibration graphs based on plasma samples spiked with the analyte were constructed by regression analysis. Typical dynamic range for standard assay was 1-2000 nmol/I. The method performance was assured by co-assaying quality control (QC) samples in duplicate at three concentration levels.

Stock and working solutions of analytes were prepared in plasma and incubated by 37° C. for 1 hour.

Sample Preparation: 40.0 μl EDTA-plasma was added 160 μl 50% methanol, 1% formic acid, then vortexed and centrifuged at 16457 g at 4° C. for 20 minutes. The supernatant was transferred to a 96 well plate, plates incubated with 0.4% BSA, 37° C. for ½ hour. Injection volume was 25 μl.

The analysis was carried out on a Sciex API 3000 mass spectrometer (MDS/Sciex, Concord, ON, Canada) using a TurbolonSpray interface. The TFC/LC system consisted of two Flux Rheos 2000 quaternary pumps, a Cohesive VIM module (Cohesive Technologies, Franklin, Mass., USA) and a CTC LC/PAL auto sampler (CTC Analytics, Zingen, Switzerland). The centrifuge employed was a HettichMikro 22R (A. Hettich, Tuttlingen, Germany). For sample clean up a TurboFlow C8 column (0.5×50 mm) (Cohesive Technologies/Thermofisher) was used and the LC separation was done on a Proteo 4 μm column (2.0×50 mm) (Phenomenex, Torrance, Calif., USA). Eluents were isocratic and gradient combinations of methanol, acetonitril, Milli-Q water and formic acid.

Non-compartmental analysis (NCA): Plasma concentration-time profiles are analyzed by non-compartmental pharmacokinetics analysis (NCA) using WinNonlin Professional 5.0 (Pharsight Inc., Mountain View, Calif., USA). NCA is performed using the individual plasma concentration-time profiles from each animal.

### Assay (III): Assay for Determining Effect on Acute Food Intake

Fasting-induced refeeding assay: Lean C57BL male mice are obtained from Charles River, Japan. They are maintained on a 12:12 light:dark cycle (lights off at 10:00 AM, lights on at 10:00 PM), fed pelleted D12450B rodent diet (Research Diets, Inc., New Brunswick, N.J.), and allowed water ad libitum. The mice arrive at 7-8 weeks of age and are acclimatized in the BioDAQ system a minimum of two weeks prior to study. On the day of study, mice are 9-12 weeks old. They are fasted overnight (20-24 h) with free access to water. On the day of the study, mice are dosed with s.c. injection (dose volume=10 mL/kg), returned to their cage, and pre-weighed food is immediately placed in the cage. The dosing vehicle used may be: 50 mM K2HPO4, 0.05% tween 80, pH=8.0 and dose is calculated for the test compound on a molar basis. Assay design: The mice are fasted from 2:00 PM the day before dosing; the mice are weighed and dosed 30 minutes before the light is turned off at 10:00 AM; the mice are dosed with 10 ml/kg s.c.; the mice are dosed once and the food-intake is monitored using the BioDAQ system (Research Diets, Inc., New Brunswick, N.J.) for 24 hours. The BioDAQ system consists of 32 mouse boxes each having a food-tray with a sensitive weight. When the mice eat the weight reduction of the content of the food-tray is registered. Data is registered each time there is a change in the weight of the individual food-tray. Cumulative food intake is calculated by subtracting the food weight at each time point from the starting food weight.

### Assay (IV): Measurement of Gastric Emptying

An exemplary assay for measurement of gastric emptying is described in the materials and methods section page 1326 under the headline “Gastric emptying” in Asakawa A et al., Characterization of the effects of pancreatic polypeptide in the regulation of energy balance, Gastroenterology, 2003, 124, 1325-1336.

### Assay (V): Measurement of Appetite

Appetite can be measured by any means known to one of skill in the art. For example, in humans, decreased appetite can be assessed by a psychological assessment. In such an aspect, administration of the receptor agonist results in a change in perceived hunger, satiety, and/or fullness. Hunger can be assessed by any means known to one of skill in the art. In some embodiments hunger is assessed using psychological assays, such as by an assessment of hunger feelings and sensory perception using e.g. a questionnaire.

### Assay (VI)—Y2 Receptor ACTOne Potency Assay

This assay provides a method for determination of in vitro effect of peptides on the Y2 receptor activity using the ACTOne based FLIPR assay. ACTOne™ is an easily scaleable cAMP biosensor HTS platform for measurement of Gs and Gi coupled 7TM receptor signalling from BD Biosciences (San Jose, Calif.). The cells express a biosensor developed around a modified rat olfactory cyclic nucleotide gated (CNG) calcium channel—a fairly non-discriminatory ion channel that responds to cAMP and cGMP. The CNG has been engineered to be cAMP selective and thus function as a cAMP responsive biosensor that signals through calcium or membrane potential responsive dyes. ACTOne HEK-293 cells expressing the Y2 receptor were obtained from BD Biosciences. The cells were loaded with a calcium responsive dye that only distributes in the cytoplasm. Probenecid, an inhibitor of the organic anion transporter was added to prevent the dye from leaving the cell. A phosphodiesterase inhibitor was added to prevent formatted cAMP from being degraded. Isoproterenol (a β1/β2 agonist) was added to activate the adenylatecyclase. When an Y2 receptor agonist was added, the adenylatecyclase was inactivated. The decreased calcium concentration in the cytoplasm was then detected as a decrease in fluorescence. Together with the test substance, isoproterenol at a concentration matching EC80 was added to all wells. The assay was carried out as follows: The cells were plated out in Greiner 384-well plates. 25 μl cell suspension containing 560 cells per μl were added to each well using the Multidrop™ (384-Multidrop from Labsystems, Finland). The cell plates were then incubated in the incubator over night at 37° C. with 5% CO2 in stacks of up to 9 plates. The cell plates were loaded with 25 μl probe from the FLIPR calcium4 kit (Molecular Devices, CA, USA) using the Multidrop™. The cell plates were returned to the incubator and incubated for 60 min at 37° C. in stacks of up to 9 plates. The cell plates were then left at room temperature for 60 min before use, without stacking the plates. The plates were covered with tinfoil to avoid light (the dye can be excited by the daylight, which results in higher baseline and variation). The FLIPR (FLIPRtetra from Molecular Devices, CA, USA) added 1 μl sample and 1 μl isoproterenol (0.05 μM final concentration) at the same time. The fluorescence signal from the wells was measured 330 seconds after sample addition on the FLIPR. The EC50 was calculated as the concentration of the Y2 receptor agonist inducing 50% decrease in fluorescence signal. A reported value of 1000 nM is intended to mean at least 1000 nM as this is the detection limit of the assay.

### Assay (VII)—Y1 Receptor ACTOne Potency Assay

This assay provides a method for determination of in vitro effect of peptides on the Y1 receptor activity using the ACTOne based FLIPR assay. The assay was carried out as described for Assay (VI) except that ACTOne HEK-293 cells expressing the Y1 receptor was used. A reported value of 1000 nM is intended to mean at least 1000 nM as this is the detection limit of the assay.

### Assay (VIII)—Y4 Receptor ACTOne Potency Assay

This assay provides a method for determination of in vitro effect of peptides on the Y1 receptor activity using the ACTOne based FLIPR assay. The assay was carried out as described for Assay (VI) except that ACTOne HEK-293 cells expressing the Y4 receptor was used. A reported value of 1000 nM is intended to mean at least 1000 nM as this is the detection limit of the assay.

### Assay (IX)—Y5 Receptor IPOne Potency Assay

The IPOne-Tb assay (Cisbio, Bagnols-sur-CèzeCedex, France) is a homogeneous time resolved fluorescence (HTRF) assay which functions as a competitive immunoassay that measures IP1 levels using cryptate labelled anti-IP1 monoclonal antibody and d2 labelled IP1, wherein IP1 is accumulated following activation of seven transmembrane receptors that couples to the Gq pathway. In the hY5 IPOne assay a HEK293 cell line stably expressing both the human Y5 receptor and the chimeric G-protein Gqi5 was used where Gqi5 ensures Gq signalling of the Gi coupled Y5 receptor. The buffers and reagents for the assay were supplied with the IPOne-Tb kit (Cisbio, Bagnols-sur-CèzeCedex, France). The assay was carried out as follows: on the day before the assay cells were seeded at a density of 40,000 cells/well in 20 μl in 384-well small volume white tissue culture plates, Greiner #784080, and incubated overnight at 37° C. with 5% CO2. On the day of the assay the media was removed and 10 μl stimulation buffer supplemented with 0.005% Tween-20 was added together with 5 μl agonist serial dilution. The plates were then incubated for 1 hour at 37° C. IP1-d2 and IP1-cryptate is reconstituted in lysis buffer according to the IPOne-Tb kit protocol. 3 μl of each of the IP1-d2 and IP1-cryptate working solutions was added to each well. The plate was incubated for 1 hour at room temperature. The plate was read on a Mithras LB 940 HTRF compatible reader (Berthold Technologies, Bad Wildbad, Germany) with 665 nm and 620 nm emission filters and the sig-nal was calculated as the fluorescence ratio 665 nm/620 nm. A reported value of 1000 nM is intended to mean at least 1000 nM as this is the detection limit of the assay.

### Assay (X)—Determination of Effect on Body Weight

Additional assays useful to the invention comprise those that can determine the effect of PP peptides on body weight and/or body composition. An exemplary assay is the following which involves utilization of a diet induced obese male C57B16J mouse model for metabolic disease: C57B16J (Taconic, Denmark) on regular diurnal rhythm and with access to a high fat diet (D12492, Research Diet, USA) are used. The mice are weighed on a weekly basis. Mice are received at age 5 weeks and put on high fat diet and housed at 24 degree celcius in normal daily rhythm. Mice are group housed 10 per cage during an obesity induction period of 14 weeks. Two weeks before study start mice are single housed (two mice per cage with a dividing wall between). One week before starting the study the mice are weighed daily to get a stable baseline and to acclimatize them to the procedure. The mice are divided into four groups (n=10/group) receiving s.c. dosing of either vehicle or compound. Dosing was performed once daily at the same time point every day, shortly before lights off. The mice are dosed for approximately 3 weeks. Body weight for all mice is recorded daily in combination with dosing. Thereafter the mice are euthanized with cervical dislocation. Data are analysed in Graph Pad Prism.

### Example 2

### In Vitro Receptor Potencies and Half-Life of PP Peptides

Receptor potency to the hY1, hY2, hY4 and hY5 receptors of PP peptides was measured using Assay (VII), (VI), (VIII) and (IX), respectively, and in vivo half-life of the PP peptides was determined in minipigs using Assay (II). The results are shown in Table 2 and 3. Crude peptide preparations had approx. 70% purity of the PP peptide.

TABLE 2
In vitro receptor potency and
half-life of PP peptides based on PP(2-36)

| | | | | | | | Half-life |
|---|---|---|---|---|---|---|---|
| | | | Receptor potency (nM) | | | | minipig |
| | | Sample | Y1, | Y2, | Y4, | Y5, | (hours), |
| Com- | Acylation | prepar- | Assay | Assay | Assay | Assay | Assay |
| pound | position | ation | (VII) | (VI) | (VIII) | (IX) | (II) |
| A | 34 | crude | 737 | >100 | 49 | 8.8 | 99 |
| B | 32 | crude | >1000 | >1000 | 143 | 38 | 93 |
| C | 31 | crude | 72 | >1000 | 3.6 | 1.0 | 94 |
| D | 30 | crude | >1000 | >1000 | 2.9 | 3.8 | 77 |
| E | 29 | crude | 36 | >1000 | 163 | 5.6 | 110 |
| F | 28 | crude | >1000 | >1000 | 1.4 | 2.5 | |
| G | 27 | crude | 254 | >1000 | 16 | 0.84 | 96 |
| H | 26 | purified | 293 | >1000 | 5.1 | 79 | 62 |
| I | 25 | purified | 259 | >1000 | >1000 | | 88 |
| J | 24 | crude | 429 | >1000 | 14 | 1.3 | 44 |
| K | 23 | crude | 129 | >1000 | 2.9 | 1.2 | 30 |
| L | 22 | crude | 26 | >1000 | 1.2 | 0.38 | 83 |
| M | 21 | crude | 67 | >1000 | 2 | 0.68 | 77 |
| N | 20 | crude | >1000 | >1000 | 67 | 2.9 | 44 |
| O | 19 | crude | 57 | >1000 | 1.8 | 0.89 | 60 |
| P | 18 | purified | 61 | 636 | 2.1 | 1.6 | 71 |
| Q | 17 | purified | 314 | >1000 | 3.9 | 5.5 | 56 |
| Q | 17 | crude | >100 | >100 | 2.0 | 0.77 | |
| R | 16 | purified | >1000 | >1000 | 3.1 | 19 | 44 |
| R | 16 | crude | >100 | >100 | 2.4 | 2.0 | 40 |
| S | 15 | crude | 76 | >1000 | 1.8 | 0.56 | 35 |
| T | 14 | crude | 81 | >1000 | 1.6 | 0.51 | 45 |
| U | 13 | crude | 214 | >1000 | 4.3 | 2.2 | 23 |
| V | 12 | crude | 555 | >1000 | 11 | 2.8 | 36 |
| W | 11 | crude | 135 | >1000 | 2.4 | 1.1 | 29 |
| X | 10 | crude | 98 | >1000 | 1.9 | 0.44 | 47 |
| Y | 9 | crude | 609 | >1000 | 7.3 | 1.9 | |
| Z | 8 | crude | 422 | >1000 | 11 | 7.7 | |
| AA | 7 | crude | 287 | >1000 | 7.1 | 0.7 | 17 |
| AB | 6 | crude | 186 | >1000 | 1.6 | 0.6 | 44 |
| AC | 5 | crude | 995 | >1000 | 34 | 3.3 | 11 |
| AD | 4 | crude | 121 | >1000 | 20 | 0.17 | 52 |
| AE | 3 | crude | 62 | >1000 | 1.6 | 0.4 | 45 |
| AF | Nα | crude | 47 | >100 | 1.0 | 1.7 | 33 |
| AF | Nα | purified | 99 | 705 | 1.2 | 46 | 36 |

TABLE 3
In vitro receptor potency and half-life of PP peptides based on PP(3-36)

| | | | Receptor potency (nM) | | | | Half-life |
|---|---|---|---|---|---|---|---|
| | | | Y1, | Y2, | Y4, | Y5, | minipig |
| | Acylation | Sample | Assay | Assay | Assay | Assay | (hours), |
| Compound | position | preparation | (VII) | (VI) | (VIII) | (IX) | Assay(II) |
| AG | 35 | crude | | | | | |
| AH | 34 | crude | >1000 | >1000 | 152 | 123 | |
| AI | 33 | crude | | | | | 45 |
| AJ | 32 | crude | >1000 | >1000 | 527 | 375 | 80 |
| AK | 31 | crude | 71 | >1000 | 6.2 | 6.4 | 80 |
| AL | 30 | crude | >1000 | >1000 | 1.8 | 33 | 59 |
| AM | 29 | crude | 47 | >1000 | 271 | 93 | 70 |
| AN | 28 | crude | >1000 | >1000 | 3.1 | 8.5 | 69 |
| AO | 27 | crude | 161 | >1000 | 29 | 10 | 44 |
| AP | 26 | crude | 300 | >1000 | 38 | 2.9 | |
| AQ | 25 | crude | 390 | >1000 | 20 | 44 | 48 |
| AR | 24 | crude | >1000 | >1000 | 16 | 6.6 | 31 |
| AS | 23 | crude | 320 | >1000 | 2.1 | 5.7 | 10 |
| AT | 22 | crude | 107 | >1000 | 1.2 | 4.1 | 34 |
| AU | 21 | crude | 184 | >1000 | 5.3 | 4.9 | 59 |
| AV | 20 | crude | >1000 | >1000 | 90 | 16 | 28 |
| AW | 19 | crude | 198 | >1000 | 2.2 | 7.7 | 33 |
| AX | 18 | purified | 664 | >1000 | 0.83 | 7.4 | |
| AY | 17 | crude | 204 | >1000 | 5.5 | 7.2 | 25 |
| AZ | 16 | crude | 864 | >1000 | 4.2 | 13 | 20 |
| BA | 15 | crude | | | | | 24 |
| BB | 14 | crude | 166 | >1000 | 2.6 | 3.9 | 19 |
| BC | 13 | crude | >1000 | >1000 | 6.7 | 17 | 16 |
| BD | 12 | crude | >1000 | >1000 | 64 | 14 | 17 |
| BE | 11 | crude | 532 | >1000 | 5.4 | | |
| BF | 10 | crude | | | | | 17 |
| BG | 9 | crude | >1000 | >1000 | 29 | 13 | |
| BH | 8 | crude | 784 | >1000 | 71 | 20 | 10 |
| BI | 7 | crude | 505 | >1000 | 30 | 5.6 | 9 |
| BJ | 6 | crude | 381 | >1000 | 6 | 4 | 29 |
| BK | 5 | crude | >1000 | >1000 | 77 | 5.8 | 54 |
| BL | 4 | crude | 181 | >1000 | 28 | >1 | 52 |
| BM | Nα | crude | 45 | >1000 | 4.2 | 6 | |
| BM | Nα | purified | 63 | >1000 | 1.3 | 10 | |

### Example 3

### Body Weight Change in DIO Mice with Compound AF

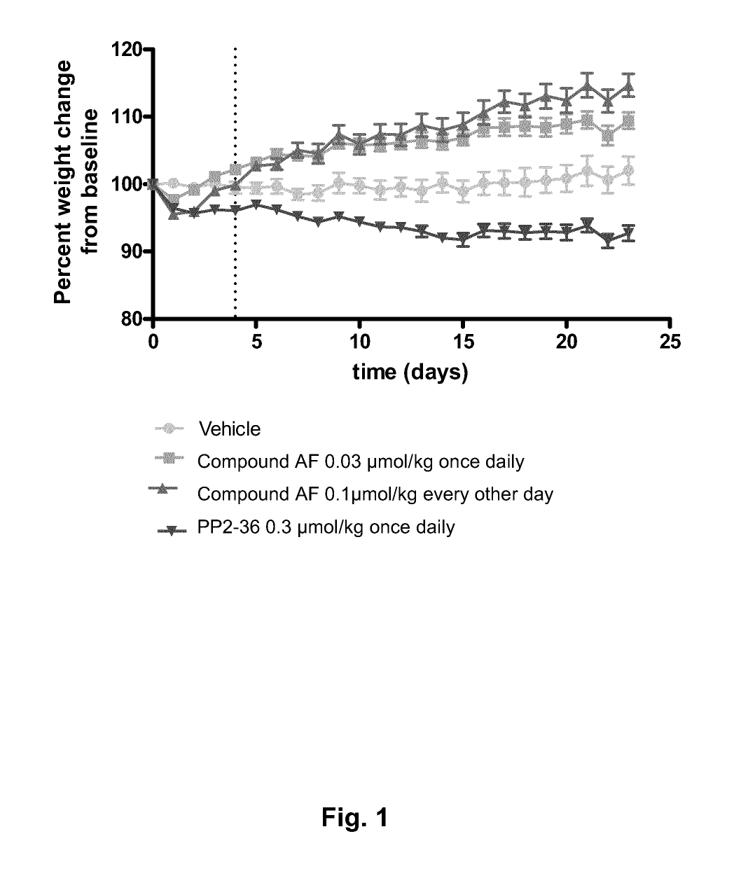

Change of body weight from baseline in male C57 DIO mice (mean±SEM, n=10) after s.c. administration of compound AF (0.03 μmol/kg once daily or 0.1 μmol/kg every other day) or the reference compound human PP(2-36) (0.3 μmol/kg, once daily) was determined using Assay (X). The results are shown in FIG. 1. At day 4, four vehicle mice dosed once with compound AF at 0.03 μmol/kg/day were excluded from the rest of the study.

The results surprisingly show an increase in body weight following administration of the acylated PP peptide while the un-acylated counterpart, PP(2-36), caused a reduction in body weight.

### Example 4

### Body Weight Change in C57Bi6J Mice with Compound BN

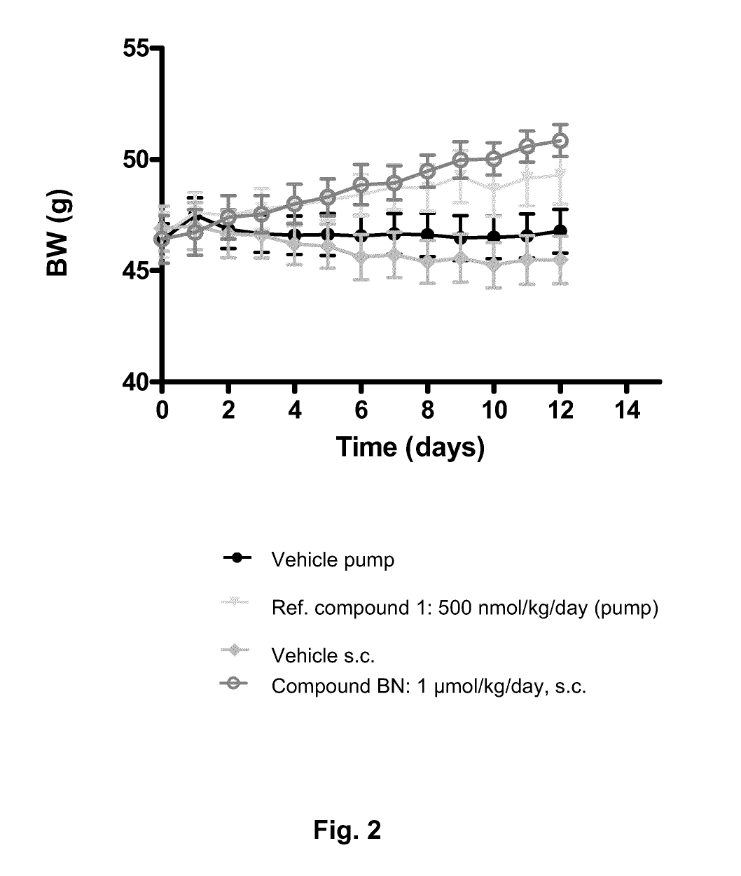

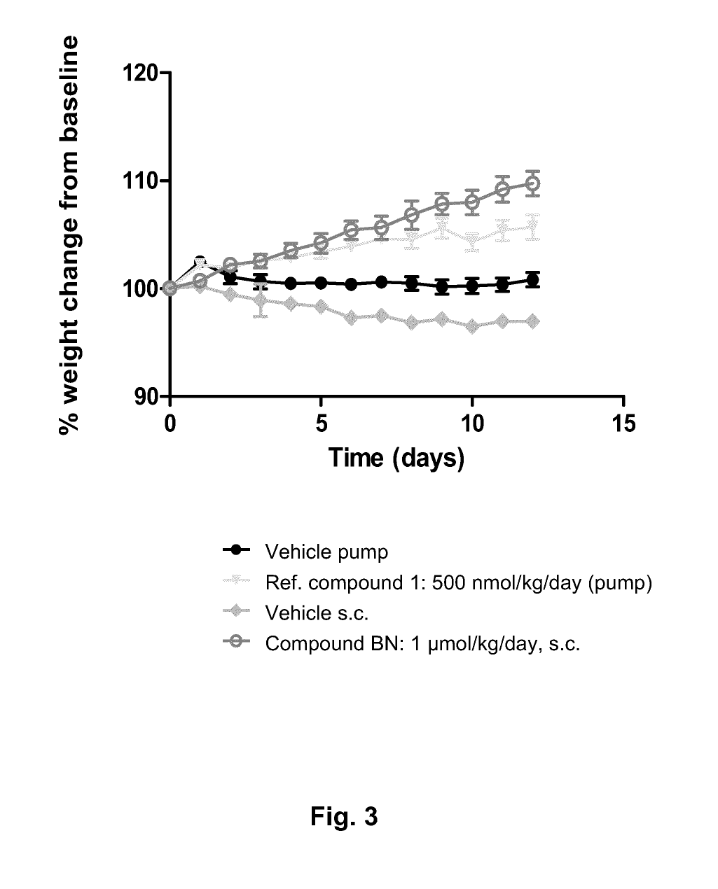

Change of body weight (mean±SEM, n=10-12) and change of body weight from baseline (mean±SEM, n=10) in male C57Bi6J mice on a high fat diet after administration of vehicle, compound BN (1 μmol/kg/day, s.c.) or reference compound 1 (500 nmol/kg/day, pump) was determined using Assay (X). The results are shown in FIGS. 2 and 3.

The results show that administration of the Y5 receptor selective PP peptide, reference compound 1, caused a minor weight gain of 4.9% compared to vehicle, whereas the acylated version of this PP peptide, i.e. compound BN, caused a markedly higher weight gain (12.7% compared to vehicle).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A human Pancreatic Polypeptide (PP) for treating and/or preventing conditions responsive to Y4 and/or Y5 receptor activation, wherein said PP peptide comprises an acylation group.

2. A PP peptide according to claim 1, wherein said treating and/or preventing provides increased food intake, increased body weight and/or increased appetite.

3. A PP peptide according to claim 1, wherein said condition is cachexia.

4. A PP peptide according to claim 1, wherein said condition is a condition characterized by damage to the intestine, such as chemotherapy-induced diarrhoea, ulcerative colitis, inflammatory bowel disease, bowel atrophy, loss bowel mucosa, and/or loss of bowel mucosal function.

5. A PP peptide according to claim 1, wherein said acylation group comprises a saturated alkyl chain with at least 14 carbon atoms, such as 16-20 carbon atoms, and wherein said alkyl chain optionally comprises a distal carboxylic acid or a distal tetrazole group.

6. A PP peptide according to claim 1, wherein said acylation group optionally comprises an 8-amino-3,6-dioxaoctanoic acid (Oeg) molecule.

7. A PP peptide according to claim 1, wherein said acylation group comprises the moiety [2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl].

8. A PP peptide according to claim 1, wherein said PP peptide comprises PP(3-36), PP(2-36), or PP(1-36), and wherein said PP(3-36), PP(2-36), or PP(1-36) comprises no more than 5 or 4, such as no more than 3, 2 or 1, amino acids substitutions, deletions and/or additions.

9. A PP peptide according to claim 1, wherein said PP peptide is selected from the group consisting of
a. PP(2-36) comprising said acylation group attached via the N-terminal amino group or any one of positions 3-9, 12-17, 19-24, 27-32 or 34;
b. PP(3-36) comprising said acylation group attached via the N-terminal amino group or any one of positions 4-35; and
c. APLEPVYPGDNATPEQLARYYKALRHYINLA-Aib-RQRQ.

10. A PP peptide according to claim 1, wherein PP peptide is selected from the group consisting of compound A to compound BM and 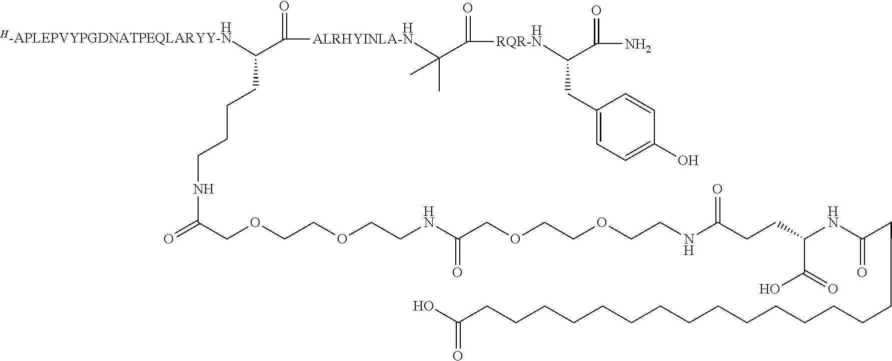
(compound BN).

11. A PP peptide according to claim 1, wherein said PP peptide
a. has a half-life of at least 2 times, such as at least 3, 4, 5 or 8 times, the half-life of PP(1-36) or wherein said PP peptide has a half-life of at least 7 h, such as at least 10, 20, 40 or 40 h, wherein the half-life is determined by Assay (II) described herein; and/or
b. has a Y4 and/or Y5 receptor potency of <100 nM, such as <50 nM, <20 nM, or <10 nM, as determined by Assay (VIII) and/or (IX), respectively.

12. A PP peptide according to claim 1, wherein a therapeutically effective dosage of said PP peptide is administered for a period of at least 2 days, such as at least 3 days or at least 4 days.

13. A PP peptide comprising an acylation group, wherein
a. said PP peptide is not PP(2-36) substituted with N-epsilon-[2-(2-{2-[2-(2-{2-[(S)-4-Carboxy-4-(17-carboxyheptadecanoylamino)butyrylamino]ethoxy}ethoxy)acetylamino]-ethoxy}ethoxy)acetyl]lysine in position 2, 10, 11, 18, 25, 26, 33, 35 or 36; or wherein
b. said PP peptide is selected from the group consisting of
i. PP(2-36) comprising said acylation group attached via the N-terminal amino group or any one of positions 3-9, 12-17, 19-24, 27-32 or 34;
ii. PP(3-36) comprising said acylation group attached via the N-terminal amino group or any one of positions 4-35; and
iii. APLEPVYPGDNATPEQLARYYKALRHYINLA-Aib-RQRQ.

14. (canceled)

15. A pharmaceutical composition comprising a PP peptide according to claim 1 and one or more pharmaceutically acceptable excipients.


SEQUENCE LISTING (3 sequences)

SEQ ID NO: 1 - protein, 36 aa, Homo sapiens
APLEPVYPGDNATPEQMAQYAADLRRYINMLTRPRQ

SEQ ID NO: 2 - protein, 36 aa, Artificial Sequence
  Analogue of PP
APLEPVYPGDNATPEQLARYYKALRHYINLAXRQRQ

SEQ ID NO: 3 - protein, 36 aa, Artificial Sequence; identical to SEQ ID NO: 2
  Analogue of PP